(19) United States
(12) Patent Application Publication
(10) Pub. No.: US 2025/0378551 A1
(43) Pub. Date: Dec. 11, 2025

(54) DIGITAL IMAGING SYSTEMS AND METHODS FOR CAPTURING 3D DATA OF AN INDIVIDUAL'S BODY

(71) Applicant: Equanimity Under Duress LLC, Santa Fe, NM (US)

(72) Inventor: Lloyd A. Yates, Chicago, IL (US)

(21) Appl. No.: 18/740,056

(22) Filed: Jun. 11, 2024

(51) Int. Cl. G06T 7/00; G06T 17/00; G16H 10/60

(52) U.S. Cl. CPC G06T 7/0012; G06T 17/00; G16H 10/60; G06T 2207/30004

(57) ABSTRACT

Digital imaging systems and methods are disclosed for detecting user-specific measurements. Digital image(s) of a user are obtained that depict one or more portions of the user's body. Body data is obtained specific to the user. A body imagery application (app) determines user-specific measurements of one or more portions of the user's body based on the digital image(s). The body imagery app determines a user-specific body-based confidence interval for the user based on the user-specific measurements and the body data. A health type-based identification of a user is generated that corresponds to one or more predefined health types based on one or more of the user-specific body-based confidence interval.

### FIELD

The present disclosure generally relates to digital imaging systems and methods, and more particularly to, digital imaging systems and methods for capturing three-dimensional (3D) data of an individual's body.

### BACKGROUND

Users are increasingly demanding means of accurately measuring their physical characteristics to measure their health and fitness and track their progress. Typically, a user will document their physical measurements with e.g., photographs, videos, journals, and must rely on their own perception to infer their health and/or fitness progress. User's may further consult friends, family, and health and/or fitness professionals, e.g., personal trainers, physicians, to inform the user of their current health or fitness status. Such consultations may happen in-person or electronically, with the in-person option being the most reliable due to electronic means being limited to two-dimensional photographs and/or videos. However, even in-person consultations are limited by the visual acuity of the individual(s) and their ability to measure and/or document the user's physical characteristics.

Accordingly, a problem arises when a user's physical characteristics imperceptible to humans may inform a health and/or fitness status of the user. This problem can lead to waste of real world assets, including fuel, labor, and time, when, for example, a user visits a doctor's office, and/or loss of life when physical characteristics indicate a health status requiring intervention. Existing technologies include sizing charts, but such charts are typically generalized such that they fail to account for the different shapes, sizes, weight distributions of different human body dimensions, and accordingly provide a false sense of fitness and/or dimensioning. Existing technologies further include three-dimensional imaging devices, however these devices are scarce, expensive, and often require a professional to operate.

For the foregoing reasons, there is a need for digital imaging systems and methods for capturing three-dimensional (3D) data of an individual's body.

### SUMMARY

Generally, as described herein, digital imaging systems and methods are described for capturing three-dimensional (3D) data of an individual's body. Such digital imaging systems and methods provide a digital imaging based solution for overcoming problems that arise from correctly identifying body measurements, and health type-based identification for specific users, each of which may have various different body dimensions, and downstream applications (uses), such as healthcare, and the like. For example, the digital imaging systems and methods described herein may be used to accurately determine sizes and proportions of physical characteristics, as determined by digital image processing, specific to a given individual.

The digital imaging systems and methods described herein may be implemented on one or more processors, either of a user computing device (e.g., such as one or more processors of a mobile device or edge device), or one or more processors of remote (cloud-based) computer or server. In some aspects, digital images may be provided to a backend server or cloud platform for image processing. However, in other aspects, image processing could be performed on an edge device.

In one example aspect, a body imagery application (app) (e.g., which may be referred to herein as the “Body Imagery” app) may be downloaded or installed on a user computing device, such as an APPLE IPHONE or GOOGLE ANDROID phone through the APPLE APP store or GOOGLE PLAY store, respectively. A user may open the app to create a user profile. Creation of the profile may include a user providing or selecting preferences, such as sizing preferences (e.g., regular, loose, slim-fit, etc.) In addition, creation of the profile may involve the user scanning himself or herself via a self-recorded video or photograph session. For example, the user can capture or take a 360-degree video to get digital images to generate user-specific measurements (e.g., where a camera is placed on ground and tilted up or otherwise angled toward the user). That is, the self-recorded video or photograph session will allow capture of one or more digital images, each of which can be used to generate or determine one or more body measurements of the user. The digital images may be Red-Green-Blue (RGB) pixel based images and/or may be Light-Detecting-and-Ranging (LiDAR) images, although other two-dimensional (2D) and/or three-dimensional (3D) images may be used, for example, including those described herein.

In various aspects approximately twenty (20) body measurements may be captured. Such body measurements serve as a foundation or basis for the user's sizing profile or otherwise user-specific measurements. In various aspects, user-specific measurements are stored in a backend server and may be accessed by the user on a respective user computing device, e.g., via a profile screen. In some aspects, the server does not store or keep any photos or videos captured by the user, thereby increasing the security and/or reducing the memory requirements of the system as a whole.

Once a user's specific measurements and/or profile information is determined, then the body imagery app may determine a user-specific body-based confidence interval (e.g., referred to herein as a “B Fit” or “Your B Fit” for a specific user). The user-specific body-based confidence interval provides one or more confidence intervals for various portions of the user's body, for example, chest, torso, arm, leg, etc.

The user-specific body-based confidence interval may be displayed via the body imagery app onto a display screen or graphic user interface (GUI) of a user's computing device.

In some aspects, the body imagery app may generate a virtual avatar of the user. In this way, the user can virtually observe their user-specific measurements.

In this way, the disclosure of the invention herein can enable user's to accurately document body measurements, their health and fitness (both presently and over time), and allow third-parties, such as physicians, to improve their ability to analyze body measurements and/or health and fitness information, both remotely and in-person.

More specifically, as described herein, a digital imaging method is disclosed for detecting user-specific body imagery. The digital imaging method comprises obtaining, by one or more processors, one or more digital images of a user. Each of the one or more digital images may depict one or more portions of the user's body. The digital imaging method further comprises obtaining, by the one or more processors, body data specific to the user. The digital imaging method further comprises determining, by a body imagery application (app) executing on the one or more processors, user-specific measurements of the one or more portions of the user's body based on the one or more digital images. The digital imaging method further comprises determining, by the body imagery app, a user-specific body-based confidence interval for the user. The user-specific body-based confidence interval may be based on the user-specific measurements and the body data. The digital imaging method further comprises generating a health type-based identification of the user based on the user-specific body-based confidence interval for one or more of the one or more portions of the user's body. The health type-based identification may be selected from one or more predefined health types.

In addition, as described herein, a digital imaging system is disclosed. The digital imaging system is configured to detect user-specific body imagery. The digital imaging system comprises a body imagery application (app) comprising computing instructions configured to execute on the one or more processors. The computing instructions of the body imagery app when executed by the one or more processors, cause the one or more processors to obtain one or more digital images of a user. Each of the one or more digital images may depict one or more portions of the user's body. The computing instructions of the body imagery app when executed by the one or more processors, may further cause the one or more processors to obtain body data specific to the user. The computing instructions of the body imagery app when executed by the one or more processors, may further cause the one or more processors to determine user-specific measurements of the one or more portions of the user's body based on the one or more digital images. The computing instructions of the body imagery app when executed by the one or more processors, may further cause the one or more processors to determine a user-specific body-based confidence interval for the user. The user-specific body-based confidence interval may be based on the user-specific measurements and the body data. The computing instructions of the body imagery app when executed by the one or more processors, may further cause the one or more processors to generate a health type-based identification of the user that corresponds to one or more predefined health types based on one or more of the user-specific body-based confidence interval.

Further, as described herein, a tangible, non-transitory computer-readable medium storing instructions for detecting user-specific body imagery is disclosed. The instructions, when executed by one or more processors, may cause the one or more processors to obtain one or more digital images of a user. Each of the one or more digital images may depict one or more portions of the user's body. The instructions, when executed by one or more processors, may further cause the one or more processors to obtain body data specific to the user. The instructions, when executed by one or more processors, may further cause the one or more processors to determine user-specific measurements of the one or more portions of the user's body based on the one or more digital images. The instructions, when executed by one or more processors, may further cause the one or more processors to determine a user-specific body-based confidence interval for the user. The user-specific body-based confidence interval may be based on the user-specific measurements and the body data. The instructions, when executed by one or more processors, may further cause the one or more processors to generate a health type-based identification of the user that corresponds to one or more predefined health types based on one or more of the user-specific body-based confidence interval.

The present disclosure relates to improvements to other technologies or technical fields at least because the present disclosure describes or introduces improvements to computing devices in the digital image processing field, whereby the digital imaging systems and methods execute on computing devices and improves the field of digital imaging, with digital based analysis of digital images of one or more digital images of a user and implementing dimensioning of such users in order to determine user-specific measurements and health type-based identification. Such systems and methods are configured to operate using a reduced processing and/or memory, and thus can operate on limited compute and memory devices, including mobile devices. For example, digital images of user (typically amounting in several megabytes or gigabytes of data) may be discarded or reduced after the user-specific measurements are generated. Such reduction frees up the computational resources of an underlying computing system, thereby making it more efficient.

Still further, the present disclosure relates to improvement to other technologies or technical fields at least because the present disclosure describes or introduces improvements to computing devices in the field of security and/or image processing, where, at least in some aspects, images of users may be preprocessed (e.g., cropped, blurred, obscured or otherwise modified) to determine user-specific measurements of a user without depicting personal identifiable information (PII) of the user (e.g., such as private areas of the user). Additionally, or alternatively, by using a virtual avatar, as described herein, a user's data can be completely abstracted from any detailed PII as shown in an original image (e.g., surface textures, skin color, birthmarks, etc. all disappear). Such features provide a security improvement, i.e., where the removal of PII (e.g., private area features) provides an improvement over prior systems because cropped or redacted images, especially ones that may be transmitted over a network (e.g., the Internet), are more secure without including PII information of an individual. Accordingly, the systems and methods described herein operate without the need for such essential information, which provides an improvement, e.g., a security improvement, over prior systems. In addition, the use of cropped, modified, or obscured images, at least in some aspects, allows the underlying system to store and/or process smaller data size images, which results in a performance increase to the underlying system as a whole because the smaller data size images require less storage memory and/or processing resources to store, process, and/or otherwise manipulate by the underlying computer system. For example, a server or other computing device need not store or keep any photos or videos taken in this digital image capture process which can thereby increase the security of the digital imaging system by removing sensitive information. For the same reason, the underlying digital imaging system is improved whereby the storage or memory resources used by the digital imaging system is condensed to the user-specific measurements (which is mere kilobytes of data for a given user) without the need to store related digital images (which would require several megabytes (MB) or gigabytes (GB) of data).

In addition, the present disclosure includes specific features other than what is well-understood, routine, conventional activity in the field, and that add unconventional steps that confine the claim to a particular useful application, e.g., digital imaging systems and methods for detecting user-specific body imagery.

Advantages will become more apparent to those of ordinary skill in the art from the following description of the preferred aspects which have been shown and described by way of illustration. As will be realized, the present aspects may be capable of other and different aspects, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts a particular aspect of the disclosed system and methods, and that each of the Figures is intended to accord with a possible aspect thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present aspects are not limited to the precise arrangements and instrumentalities shown, wherein:

FIG. 1 illustrates an example digital imaging system configured to detect user-specific body imagery, in accordance with various aspects disclosed herein.

FIG. 2 illustrates an example digital imaging method for detecting user-specific body imagery, in accordance with various aspects disclosed herein.

FIG. 3 illustrates an example graphic user interface (GUI) as rendered on a display screen of a user computing device regarding user-specific measurements in accordance with various aspects disclosed herein.

FIG. 4 illustrates an example graphic user interface (GUI) as rendered on a display screen of a user computing device regarding body data in accordance with various aspects disclosed herein.

FIG. 5 illustrates an example graphic user interface (GUI) as rendered on a display screen of a user computing device regarding virtual avatar(s) in accordance with various aspects disclosed herein.

FIG. 6 illustrates an example graphic user interface (GUI) as rendered on a display screen of a user computing device regarding a user-specific body-based confidence interval in accordance with various aspects disclosed herein.

The Figures depict preferred aspects for purposes of illustration only. Alternative aspects of the systems and methods illustrated herein may be employed without departing from the principles of the invention described herein.

### DETAILED DESCRIPTION OF THE INVENTION

The present disclosure relates to digital imaging systems and methods for detecting user-specific body imagery. Such systems and methods comprise analyzing or scanning a user's body in order to obtain digital images that are then used to generate a health type-based identification of a user that corresponds to one or more predefined health types based on one or more of the user-specific body-based confidence interval. The digital images may be used to determine a user's specific size and dimensions based on the processing of one or more digital images of the user to determine the physical attributes of the user.

Generally, user-specific measurements of the one or more portions of the user's body may be determined from one or more digital images of a user (e.g., digital image 202a as describe herein). In some aspects, the digital images may be two-dimensional (2D). Additionally, or alternatively, the digital images may be three-dimensional (3D) or contain three-dimensional data. The digital images may additionally or alternatively comprise 2D and/or 3D scans (e.g., where a computing includes a scanning function or capability), comprising respective 2D and/or 3D data of such scans. In various aspects, the digital image(s) (e.g., digital 202a) may comprise various data types and/or formats as captured by various 2D and/or 3D imaging capture systems or cameras, including, by way of non-limiting example, light-detecting-and-ranging (LiDAR) based digital images, time-of-flight (ToF) based digital images, other similar types of images as captured by 2D and/or 3D imaging capture systems and/or cameras. Compared to LiDAR, typical implementations of ToF image analysis involves a similar, but different, creation “depth maps” based on light detection, usually through a standard RGB camera. With respect to the disclosure herein, LiDAR, ToF, and/or other 3D imaging techniques are compatible, and may each, together or alone, be used with, the disclosure and/or aspects herein. In various aspects, such digital images may be saved or stored in formats, including, but not limited to, e.g., JPG, TIFF, GIF, BMP, PNG, and/or other files, data types, and/or formats for saving or storing such images.

In addition, such digital images may comprise color and/or channel data, including by way of non-limiting example, red-green-blue (RGB) data, CIELAB (LAB) data, hue saturation value (HSV) data, and/or or other color formats and/channels. Such digital images may be captured, stored, processed, analyzed, and/or otherwise manipulated and used as described herein, by body imagery application, digital imaging system 100, or otherwise as described herein.

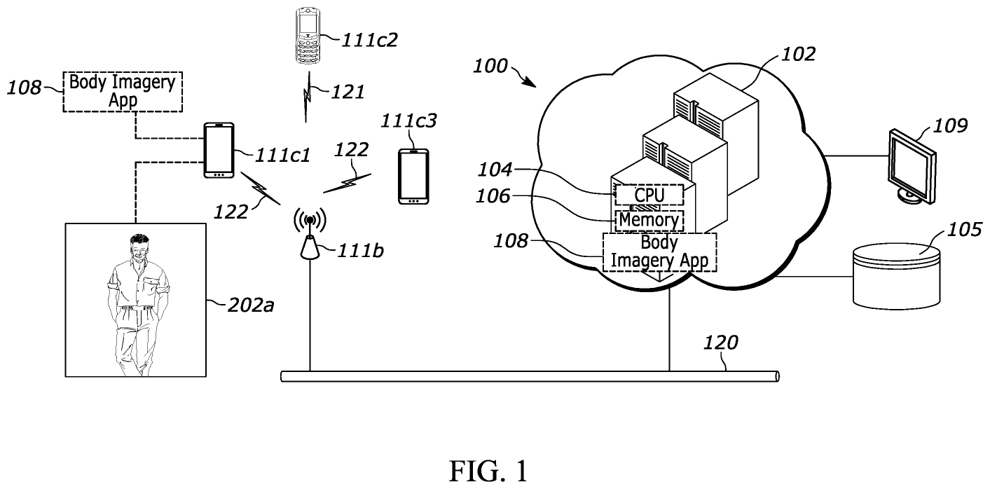

FIG. 1 illustrates an example digital imaging system 100 configured to detect user-specific measurements, in accordance with various aspects disclosed herein. In the example aspect of FIG. 1, digital imaging system 100 includes server(s) 102, which may comprise one or more computer servers. In various aspects server(s) 102 comprise multiple servers, which may comprise multiple, redundant, or replicated servers as part of a server farm. In still further aspects, server(s) 102 may be implemented as cloud-based servers, such as a cloud-based computing platform. For example, imaging server(s) 102 may be any one or more cloud-based platform(s) such as MICROSOFT AZURE, AMAZON AWS, or the like. Server(s) 102 may include one or more processor(s) 104 as well as one or more computer memories 106. In various aspects, server(s) 102 may be referred to herein as “imaging server(s).”

Memories 106 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 106 may store an operating system (OS) (e.g., Microsoft Windows, Linux, UNIX, etc.) capable of facilitating the functionalities, apps, methods, or other software as discussed herein. Memorie(s) 106 may also store a body imagery application (app) 108, a remote app, for capturing and/or analyzing digital images (e.g., digital image 202a), as described herein. Additionally, or alternatively, digital images, such as digital image 202a, may also be stored in database 105, which is accessible or otherwise communicatively coupled to imaging server(s) 102. In addition, memories 106 may also store machine readable instructions, including any of one or more application(s) (e.g., an imaging application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. It should be appreciated that one or more other applications may be envisioned and that are executed by the processor(s) 104. It should be appreciated that given the state of advancements of mobile computing devices, all of the processes functions and steps described herein may be present together on a mobile computing device (e.g., user computing device 111c1). In some aspects, memorie(s) 104 may store a health identification artificial intelligence (AI) model and/or Machine Learning (ML) model, as described herein.

The processor(s) 104 may be connected to the memories 106 via a computer bus responsible for transmitting electronic data, data packets, or otherwise electronic signals to and from the processor(s) 104 and memories 106 in order to implement or perform the machine readable instructions, methods, processes, elements or limitations, as illustrated, depicted, or described for the various flowcharts, illustrations, diagrams, figures, and/or other disclosure herein.

Processor(s) 104 may interface with memory 106 via the computer bus to execute an operating system (OS). Processor(s) 104 may also interface with the memory 106 via the computer bus to create, read, update, delete, or otherwise access or interact with the data stored in memories 106 and/or the database 105 (e.g., a relational database, such as Oracle, DB2, MySQL, or a NoSQL based database, such as MongoDB). The data stored in memories 106 and/or database 105 may include all or part of any of the data or information described herein, including, for example, digital images (e.g., digital image 202a), user-specific measurements, user profile information, and/or other images and/or information such as or the like, or as otherwise described herein.

Imaging server(s) 102 may further include a communication component configured to communicate (e.g., send and receive) data via one or more external/network port(s) to one or more networks or local terminals, such as computer network 120 and/or terminal 109 (for rendering or visualizing) described herein. In some aspects, imaging server(s) 102 may include a client-server platform technology such as ASP.NET, Java J2EE, Ruby on Rails, Node.js, a web service or online API, responsive for receiving and responding to electronic requests. The imaging server(s) 102 may implement the client-server platform technology that may interact, via the computer bus, with the memories(s) 106 (including the applications(s), component(s), API(s), data, etc. stored therein) and/or database 105 to implement or perform the machine readable instructions, methods, processes, elements or limitations, as illustrated, depicted, or described for the various flowcharts, illustrations, diagrams, figures, and/or other disclosure herein.

In various aspects, the imaging server(s) 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some aspects, computer network 120 may comprise a private network or local area network (LAN). Additionally, or alternatively, computer network 120 may comprise a public network such as the Internet.

Imaging server(s) 102 may further include or implement an operator interface configured to present information to an administrator or operator and/or receive inputs from the administrator or operator. As shown in FIG. 1, an operator interface may provide a display screen (e.g., via terminal 109). Imaging server(s) 102 may also provide I/O components (e.g., ports, capacitive or resistive touch sensitive input panels, keys, buttons, lights, LEDs), which may be directly accessible via, or attached to, imaging server(s) 102 or may be indirectly accessible via or attached to terminal 109. According to some aspects, an administrator or operator may access the server 102 via terminal 109 to review information, make changes, and/or perform other functions as described herein.

In some aspects, imaging server(s) 102 may perform the functionalities as discussed herein as part of a “cloud” network or may otherwise communicate with other hardware or software components within the cloud to send, retrieve, or otherwise analyze data or information described herein.

In general, a computer program or computer based product, application, or code (e.g., the app or computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 104 (e.g., working in connection with the respective operating system in memories 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

In some aspects, at least one of a plurality of machine learning (ML) methods and algorithms may be applied by one or more modules which may include, but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various aspects, the implemented ML methods and algorithms are directed toward at least one of a plurality of categorizations of ML, such as supervised learning, unsupervised learning, and reinforcement learning. The one or more modules may be stored, for example, on memorie(s) 106 and/or database 105.

In certain aspects, the ML based algorithms may be included as a library or package executed on server(s) 102. For example, libraries may include the TensorFlow based library, the Pytorch library, and/or the scikit-learn Python library.

In various aspects, an ML module may employ supervised learning, which involves identifying patterns in existing data to make predictions about subsequently received data. Specifically, the ML module may be “trained” using training data, which includes example inputs and associated example outputs. Based upon the training data, the ML module may generate a predictive function which maps outputs to inputs and may utilize the predictive function to generate ML outputs based upon data inputs. The exemplary inputs and exemplary outputs of the training data may include any of the data inputs or ML outputs described above. In exemplary embodiments, a processing element may be trained by providing it with a large sample of data with known characteristics or features.

In some aspects, the ML module may employ unsupervised learning, which involves finding meaningful relationships in unorganized data. Unlike supervised learning, unsupervised learning does not involve user-initiated training based upon example inputs with associated outputs. Rather, in unsupervised learning, the ML module may organize unlabeled data according to a relationship determined by at least one ML method/algorithm employed by the ML module. Unorganized data may include any combination of data inputs and/or ML outputs as described above.

In certain aspects, the ML module may employ reinforcement learning, which involves optimizing outputs based upon feedback from a reward signal. Specifically, the ML module may receive a user-defined reward signal definition, receive a data input, utilize a decision-making model to generate the ML output based upon the data input, receive a reward signal based upon the reward signal definition and the ML output, and alter the decision-making model so as to receive a stronger reward signal for subsequently generated ML outputs. Other types of ML may also be employed, including deep or combined learning techniques.

The ML module may receive labeled data at an input layer of a model having a networked layer architecture (e.g., an artificial neural network, a convolutional neural network, etc.) for training the one or more ML models. The received data may be propagated through one or more connected deep layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models. The output layer may be trained to output a prediction, for example.

The ML module may comprise a set of computer-executable instructions implementing ML loading, configuration, initialization and/or operation functionality. The ML module may include instructions for storing trained models (e.g., in the database 105). As discussed, once trained, the one or more trained ML models may be operated in inference mode, whereupon when provided with de novo input that the model has not previously been provided, the model may output one or more predictions, classifications, etc., as described herein.

As shown in FIG. 1, imaging server(s) 102 are communicatively connected, via computer network 120 to the one or more user computing devices 111c1-111c3 via base station 111b. In some aspects, base station 111b comprise a cellular base station, such as a cell tower, communicating to the one or more user computing devices 111c1-111c3 via wireless communications 121 based on any one or more of various mobile phone standards, including NMT, GSM, CDMA, UMMTS, LTE, 5G, or the like. Additionally, or alternatively, base stations 111b may comprise routers, wireless switches, or other such wireless connection points communicating to the one or more user computing devices 111c1-111c3 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c3 may comprise mobile devices and/or client devices for accessing and/or communications with imaging server(s) 102. Such mobile devices may comprise one or more mobile processor(s) and/or an imaging device for capturing images, such as images as described herein (e.g., digital image 202a). In various aspects, user computing devices 111c1-111c3 may comprise a mobile phone (e.g., a cellular phone), a tablet device, a personal data assistance (PDA), or the like, including, by non-limiting example, an APPLE IPHONE or IPAD device or a GOOGLE ANDROID based mobile phone or tablet.

In various aspects, the one or more user computing devices 111c1-111c3 may implement or execute an operating system (OS) or mobile platform such as APPLE IOS and/or Google ANDROID operation system. Any of the one or more user computing devices 111c1-111c3 may comprise one or more processors and/or one or more memories for storing, implementing, or executing computing instructions or code, e.g., a mobile application, as described in various aspects herein. As shown in FIG. 1, body imagery app 108 and/or an imaging application (e.g., as described herein), or at least portions thereof, may also be stored locally on a memory of a user computing device (e.g., user computing device 111c1).

User computing devices 111c1-111c3 may comprise a wireless transceiver to receive and transmit wireless communications 121 and/or 122 to and from base station 111b. In various aspects, digital images (e.g., digital image 202a) may be transmitted via computer network 120 to imaging server(s) 102 for imaging analysis as described herein.

In addition, the one or more user computing devices 111c1-111c3 may include a digital camera, digital video camera, and/or otherwise imaging capture device or system for capturing or taking digital images and/or frames (e.g., digital image 202a). Each digital image may comprise LiDAR, ToF, and/or pixel data, which may be used for imaging analysis as described herein. For example, a digital camera and/or digital video camera of, e.g., any of user computing devices 111c1-111c3 may be configured to take, capture, or otherwise generate digital images (e.g., digital image 202a) and, at least in some aspects, may store such images in a memory of a respective user computing devices. Additionally, or alternatively, such digital images may also be transmitted to and/or stored on memorie(s) 106 and/or database 105 of server(s) 102.

Still further, each of the one or more user computer devices 111c1-111c3 may include a display screen for displaying graphics, images, text, dimension(s), virtual avatars, data, pixels, features, and/or other such visualizations or information as described herein. In various aspects, graphics, images, text, dimension(s), product sizes, data, pixels, features, and/or other such visualizations or information may be received from imaging server(s) 102 for display on the display screen of any one or more of user computer devices 111c1-111c3. Additionally, or alternatively, a user computer device may comprise, implement, have access to, render, or otherwise expose, at least in part, an interface or a graphic user interface (GUI) for displaying text and/or images on its display screen. In various aspects, a display screen can also be used for providing information, instructions, and/or guidance to the user of a given device (e.g., user computing device 111c1).

In some aspects, computing instructions and/or applications executing at the server (e.g., server(s) 102) and/or at a mobile device (e.g., mobile device 111c1) may be communicatively connected for analyzing LiDAR data, ToF data, and/or pixel data of one or more digital images depicting users and/or related user-specific measurements, as described herein. For example, one or more processors (e.g., processor(s) 104) of server(s) 102 may be communicatively coupled to a mobile device via a computer network (e.g., computer network 120). In such aspects, a body imagery app may comprise a server app portion configured to execute on the one or more processors of the server (e.g., server(s) 102) and a mobile app portion configured to execute on one or more processors of the mobile device (e.g., any of one or more user computing devices 111c1-111c3) and/or other such standalone imaging device. In such aspects, the server app portion is configured to communicate with the mobile app portion. The server app portion or the mobile app portion may each be configured to implement, or partially implement, one or more of: (1) obtaining, by one or more processors, one or more digital images of a user (e.g., digital image 202a), each of the one or more digital images depicting one or more portions of the user's body; (2) obtaining, by the one or more processors, body data specific to the user; (3) determining, by a body imagery application (app) executing on the one or more processors, user-specific measurements of the one or more portions of the user's body based on the one or more digital images; (4) determining, by the body imagery app, a user-specific body-based confidence interval for one or more of the one or more portions of the user's body, the user-specific body-based confidence interval based on the user-specific measurements and the body data; and (5) generating a health type-based identification of the user that corresponds to one or more predefined health types based on one or more of the user-specific body-based confidence interval.

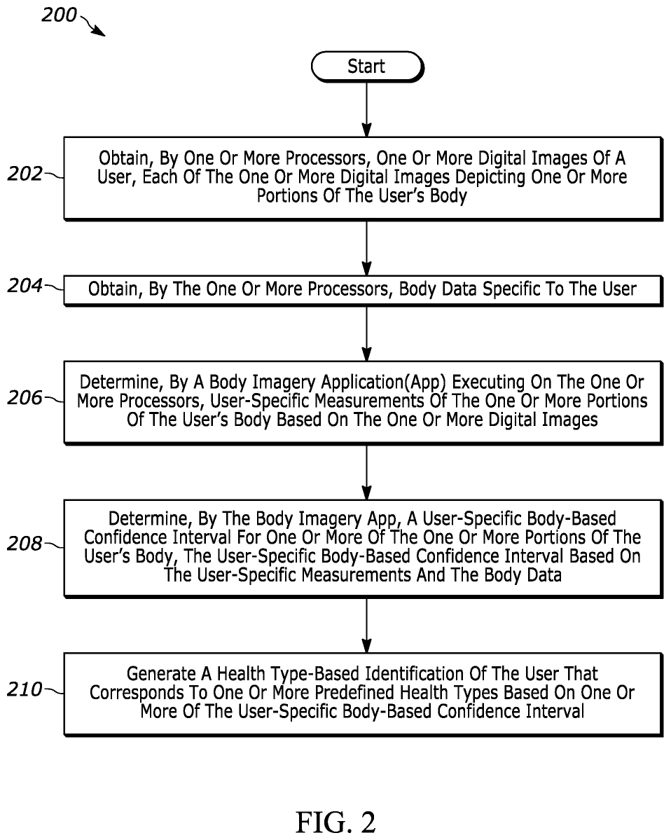

FIG. 2 illustrates an example digital imaging method 200 for detecting user-specific body imagery, in accordance with various aspects disclosed herein. At block 202, digital imaging method 200 comprises obtaining, by one or more processors (e.g., such as a processor of user computing device 111c1), one or more digital images (e.g., such as a self-recorded video) of a user. Each of the one or more digital images may depict one or more portions of the user's body.

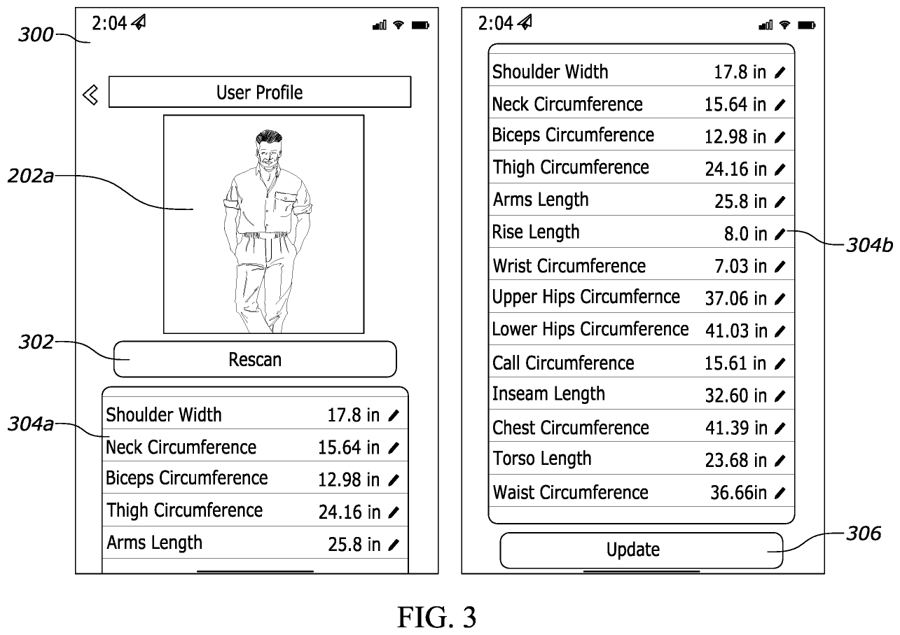

For example, in various aspects, body imagery app, executing on one or more processors of a user computing device 111c1, can capture or scan a user to obtain a recorded video or one or more digital images of the user (e.g., digital image 202a). The digital image(s) may then be used to generate body measurements (e.g., for example, approximately 20 measurements as shown for FIG. 3 herein). In various aspects, the server (e.g., server 102) need not store or keep any photos or videos taken in this digital image capture process. By implementing this aspect, the security of the digital imaging system is increased by avoiding storage of sensitive user information. For the same reason, the underlying digital imaging system is improved whereby the storage or memory resources used by the digital imaging system is condensed to the user-specific measurements (which is mere kilobytes of data for a given user) without the need to store related digital images (which would require several megabytes (MB) or gigabytes (GB) of data per user).

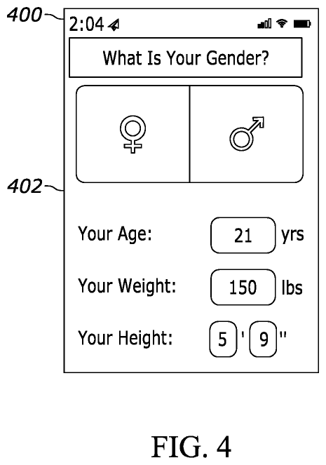

With further reference to FIG. 2, at block 204, digital imaging method 200 further comprises obtaining, by the one or more processors, body data specific to the user. Body data may comprise weight and height information specific to the user, as well as user preferences, although it is to be understood that other information may comprise body data of a user. For example, in various aspects, body imagery app can request that a user create a profile, where the user inputs information regarding his or her body dimensions, preferences, and the like. Body data is further described herein with respect to FIG. 4.

At block 206, digital imaging method 200 further comprises determining, by the body imagery app, executing on the one or more processors (e.g., one or more processors of user computing device 111c1 and/or server 102), user-specific measurements of the one or more portions of the user's body based on the one or more digital images (e.g., digital image 202a). Such body measurements may be used to generate or otherwise determine a user's sizing profile or user-specific measurements. In some aspects, such the user's sizing profile or user-specific measurements may be stored in a backend server (e.g., server 102), and may be accessible to the user via the body imagery app, for example, via their profile view as illustrated by FIG. 3 herein. The sizing profile (e.g., comprising user-specific measurements) are described further herein for FIG. 3.

In some aspects, a user profile of the user may be generated for the user. The user profile may include or otherwise be based on one or more digital images as captured for the user, body data of the user, or other user-specific information as described herein. For example, the body imagery app can request a user to create a profile, involving the user to scan himself or herself through a self-recorded video and/or digital imaging. In one aspect, as described for FIG. 3, such scanning can generate approximately 20 body measurements, which can serve as the foundation to a user's sizing profile. This information can be incorporated into the user's profile along with other information as provided by the user or as determined for the user via the body imagery app based on the digital images. Additionally, or alternatively, a user's profile may comprise e.g., user-specific body-based confidence intervals, health type-based identification, health report, etc.

In some aspects, a user may initiate electronic transmission of the user profile to a second user. The user profile may, for example be transmitted from the body imagery app and/or from the server(s) (e.g., server 102) where it is stored. Once received, the user profile allows a second user to observe and/or evaluate the user based on the user's profile. This allows sharing the user profile with friends, family, and/or otherwise a third-party user. That is, in such aspects, each user is able to share and store sizing profiles of their friends, family, and significant others on the app and/or servers, allowing loved ones to know exact sizing and measurements for the user and/or a health type-based identification. In addition, a user may have multiple user profiles defining different sizing profiles or otherwise user-specific measurements. The user then may share a specific user profile which allows the user to provide friends, family, and/or third parties with confidence and knowledge of the user's exact size and e.g., health type-based identification for a specific user profile.

In some aspects, a third party may include any individual, entity, or group which a user may desire to directly and/or indirectly share their user profile with. For example, a third party may include medical professionals (e.g., physicians), medical entities (e.g., hospitals), insurance entities (e.g., health insurance provider, life insurance provider), technology entities (e.g., APPLE, GOOGLE, etc.), etc.

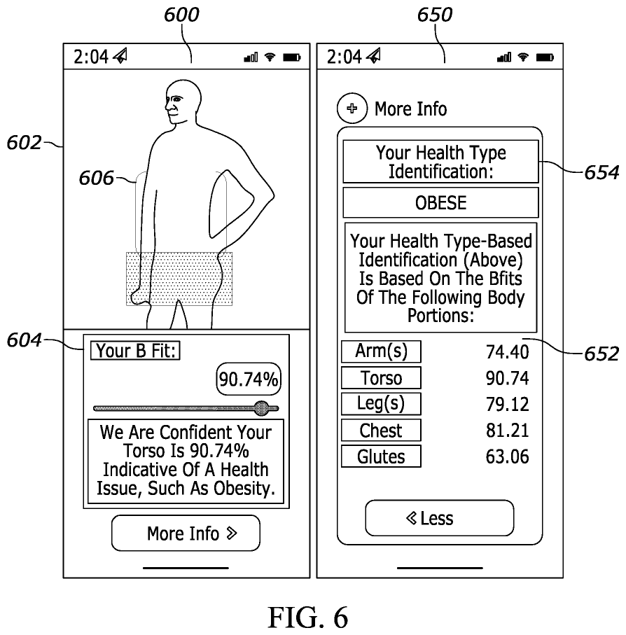

With further reference to FIG. 2, at block 208, digital imaging method 200 further comprises determining, by the body imagery app, a user-specific body-based confidence interval for the user. The user-specific body-based confidence interval may also be referred to as a user's “B Fit.” In various aspects, the user-specific body-based confidence interval is based on the user-specific measurements and the body data. In various aspects, the user-specific body-based confidence interval provides percentile classifications or indications that can represent a body portion indicating a multitude of health issue(s). Aspects of the user-specific body-based confidence interval are further described herein with respect to FIG. 6.

At block 210, digital imaging method 200 further comprises generating a health type-based identification of the user that corresponds to one or more predefined health types based on one or more of the user-specific body-based confidence interval. A health type-based identification and predefined health types are further described herein with respect to FIG. 6. A health type-based identification may have displayed therewith, on a GUI of the user computing device, a user-specific body-based confidence interval, i.e., the user's “B Fit,” as further described herein with respect to FIG. 6.

FIG. 3 illustrates an example graphic user interface (GUI) as rendered on a display screen 300 of a user computing device (e.g., user computing device 111c1) regarding user-specific measurements in accordance with various aspects disclosed herein. For example, as shown in the example of FIG. 3, GUI 300 may be implemented or rendered by the body imagery app on a display screen via of a user computing device (e.g., user computing device 111c1). For example, as shown in the example of FIG. 3, GUI 300 may be implemented or rendered via a native app executing on user computing device 111c1. In the example of FIG. 3, user computing device 111cl is a user computer device as described for FIG. 1, e.g., where 111c1 is illustrated as an APPLE IPHONE that implements the APPLE IOS operating system and that has a display screen. User computing device 111c1 may execute one or more native applications (apps) on its operating system, including, for example, the body imagery app 108 as described herein. Such native apps may be implemented or coded (e.g., as computing instructions) in a computing language (e.g., SWIFT) executable by the user computing device operating system (e.g., APPLE iOS) by the processor of user computing device 111c1.

Additionally, or alternatively, GUI 300 may be implemented or rendered via a web interface, such as via a web browser application, e.g., Safari and/or Google Chrome app(s), or other such web browser or the like.

FIG. 3 illustrates a user's sizing profile or otherwise user-specific measurements. The example of FIG. 3 may comprise a completed sizing profile, which may be based on one or more images as captured of the user. One such image may be image 202a, which depicts a front portion of a user.

As illustrated for FIG. 3, the user's sizing profile comprises user-specific measurements. These user-specific measurements include body measurements of the user as determined from the one or more digital images (as described for the algorithm of digital imaging method 200). Such measurements and measurement values may include, as shown for GUI portions 304a and 304b of GUI 300, and by way of non-limiting example, a user's shoulder width (e.g., 17.8 inches), neck circumference (e.g., 15.64 inches), biceps circumference (e.g., 12.96 inches), thigh circumference (e.g., 24.16 inches), arms length (e.g., 25.8 inches), rise length (e.g., 8.0 inches), wrist circumference (e.g., 7.03 inches), upper hips circumference (e.g., 37.06 inches), lower hips circumference (e.g., 41.03 inches), calf circumference (e.g., 15.61 inches), inseam length (e.g., 32.69 inches), chest circumference (e.g., 41.39 inches), torso length (e.g., 23.66 inches), and waist circumference (e.g., 36.66 inches). It is to be understood, however, that addition and/or different measurements and measurement values for a user's user-specific measurements are contemplated herein, and each of which may be determined using the digital imaging systems and methods as described herein.

In some aspects, the user-specific measurements of the one or more portions of the user's body comprise one or more of the following: width, height, length, circumference, volume. For example, a user's user-specific measurements may include a total height of 170 centimeters, and a volume of 0.062 cubic meters.

As described for FIG. 3, digital image 202a depicts a front portion of a user. In the example of FIG. 3, digital image 202a is represented as a 2D pixel based image. However, as described herein, digital image(s) may be captured in one or both of 2D and/or 3D images and used to determine the user's user-specific measurements. In various aspects, digital image 202a may each be digital images as captured by a digital camera or otherwise digital imaging capture devices, as described for FIG. 1. Digital image 202a may be transmitted to and from server(s) 102 via a user computer device (e.g., user computing devices 111c1), computer network 120, and base station 111b, as shown and described for FIG. 1.

In addition, in various aspects, a digital image (e.g., digital image 202a) may comprise pixel data (e.g., RGB data) comprising feature data and corresponding to one or more image features, within the respective image. The pixel data may be captured by an imaging device of one of the user computing devices (e.g., one or more user computer devices 111c1-111c3). For example, with respect to digital images as described herein, pixel data (e.g., pixel data of the digital image 202a) may comprise individual points or squares of data within an image, where each point or square represents a single pixel within an image. Each pixel may be at a specific location within an image. In addition, each pixel may have a specific color (or lack thereof). Pixel color, may be determined by a color format and related channel data associated with a given pixel. For example, one color format includes the red-green-blue (RGB) format having red, green, and blue channels. That is, in the RGB format, data of a pixel is represented by three numerical RGB components (Red, Green, Blue), that may be referred to as a channel data, to manipulate the color of pixel's area within the image. In some implementations, the three RGB components may be represented as three 8-bit numbers for each pixel. Three 8-bit bytes (one byte for each of RGB) may be used to generate 24-bit color. Each 8-bit RGB component can have 256 possible values, ranging from 0 to 255 (i.e., in the base 2 binary system, an 8-bit byte can contain one of 256 numeric values ranging from 0 to 255). This channel data (R, G, and B) can be assigned a value from 0 to 255 that can be used to set the pixel's color. For example, three values like (250, 165, 0), meaning (Red=250, Green=165, Blue=0), can denote one Orange pixel. As a further example, (Red=255, Green=255, Blue=0) means Red and Green, each fully saturated (255 is as bright as 8 bits can be), with no Blue (zero), with the resulting color being Yellow. As a still further example, the color black has an RGB value of (Red=0, Green=0, Blue=0) and white has an RGB value of (Red=255, Green=255, Blue=255). Gray has the property of having equal or similar RGB values, for example, (Red=220, Green=220, Blue=220) is a light gray (near white), and (Red=40, Green=40, Blue=40) is a dark gray (near black).

In this way, the composite of three RGB values creates a final color for a given pixel. With a 24-bit RGB color image, using 3 bytes to define a color, there can be 256 shades of red, and 256 shades of green, and 256 shades of blue. This provides 256×256×256, i.e., 16.7 million possible combinations or colors for 24 bit RGB color images. As such, a pixel's RGB data value indicates a degree of color or light each of a Red, a Green, and a Blue pixel is comprised of. The three colors, and their intensity levels, are combined at that image pixel, i.e., at that pixel location on a display screen, to illuminate a display screen at that location with that color. It is to be understood, however, that other bit sizes, having fewer or more bits, e.g., 10-bits, may be used to result in fewer or more overall colors and ranges. Further, it is to be understood that the pixel data may contain additional or alternative color format and channel data. For example, the pixel data may include color data expressed in a hue saturation value (HSV) format or hue saturation lightness (HSL) format.

As a whole, the various pixels, positioned together in a grid pattern form a digital image or portion thereof. A single digital image can comprise thousands or millions of pixels or channels. Images can be captured, generated, stored, and/or transmitted in a number of formats, such as JPEG, TIFF, PNG and GIF. These formats use pixels to store or represent the image.

With reference to FIG. 3, digital image 202a illustrates a user. Digital image may comprise a plurality of pixels. The pixel data, and features thereof, may indicate body dimensions, sizes, apparel dimensions, etc. of the user that maybe be used to determine user-specific measurements. For example, pixels may define features determined from or otherwise based on one or more pixels in a digital image. For example, with respect to image 202a, pixels forming the user's arms may be used to determine arm circumference and those pixels that form the user's waist may determine waist circumference. The identified pixels may be determined as those pixels grouped together that form a shape of an arm or waist such that the body imagery app may correctly scan and categorize each measurement. Moreover, an edge of a user's body may be determined by an abrupt change in RGB values indicating that the neighboring pixels belong to two different areas of the body. A collection of surface edges can be used to determine a body outline, and the position of those edges relative to other parts of the body can be used to determine which body part has been located (e.g., a finger should be attached to a hand, which should be attached to an arm, etc.). The capture and scanning of multiple images (e.g., a 360 view of the user for obtaining a plurality of images) can be used to enhance the accuracy of the user-specific measurements.

In some aspects, one or more portions of the user's body comprises a first body portion and a second body portion, and the user-specific body-based confidence interval is based on a proportion identified within the pixel data between the first body portion and the second body portion. For example, surface edges of two body portions (i.e., body parts) may be determined based on an abrupt change in RGB values indicating that the neighboring pixels belong to two different areas of the body. The user-specific body-based confidence interval may be based on the pixel data of each of the two body portions by, for example, comparing the user-specific measurement (e.g., width of right forearm) determined from the surface edges of the first body portion (e.g., right forearm) to the user-specific measurement (e.g., width of right upper arm) determined from the surface edges of the second body portion (e.g., right upper arm).

Additionally, or alternatively, in some aspects, the user-specific body-based confidence interval is based on (i) a proportion identified within the pixel data between the first body portion and the second body portion, and (ii) the body data. Continuing the previous example, fitness information of body data may indicate a user has a bench press personal record of 500 pounds. The user-specific body-based confidence interval may be based on a proportion identified within the pixel data between the first body portion (e.g., right forearm) and the second body portion (e.g., right upper arm), and (ii) the fitness information of the body data indicating a user has a bench press personal record of 500 pounds. In this example, the proportion of a right forearm to a right upper arm may indicate downstream a health type-based identification of “obese”, however, when body data is considered, a proportion of a right forearm to a right upper arm may not indicate downstream a health type-based identification of “obese.”

In some aspects, the one or more portions of the user's body comprises a first body portion, and the user-specific body-based confidence interval is based on a proportion identified within the pixel data between the first body portion and one or more of the one or more portions of the user's body. For example, the user-specific body-based confidence interval may be based on a proportion of a first body portion, such as a neck circumference, to one or more body portions, such as chest circumference, total height, arm length, etc.

As further shown for FIG. 3, a user can select from GUI 300 to rescan 302 their body in order to generate new user-specific measurements. Similarly, as shown for GUI 300 a user may similarly select to update 306 the user-specific measurements. A rescan 302 may include a full scan for generation of all new user-specific measurements. An update 306 may include updating one or more user-specific measurements. For example, if a user clicks on rescan 302, body imagery app may prompt the user with a series of screens with instructions on how to record a one or more digital images (e.g., a video) in order to correctly to obtain accurate user-specific measurements, as shown in FIG. 3 for GUI portions 304a and 304b of GUI 300.

In various aspects, the user-specific measurements may be rendered on GUI 300 in about 60 seconds (or less) after obtaining the digital images from the scan. In some aspects, a user may capture new digital images that may be transmitted to imaging server(s) 102 for determination and/or storage of user-specific measurements. In other aspects, the new digital images may be locally obtained by computing device 111cl and analyzed for user-specific measurements on the computing device 111c1.

FIG. 4 illustrates an example graphic user interface (GUI) 400 as rendered on a display screen of a user computing device regarding body data in accordance with various aspects disclosed herein. GUI 400 may be implemented or rendered in the same manner as described herein for GUI 300 of FIG. 3, where, for example, the body imagery app renders GUI 400 on a display screen via of a user computing device (e.g., user computing device 111c1).

As illustrated for FIG. 4, body data 402 comprises user specific information such as sex (e.g., male or female), age (e.g., 21 years old), weight (e.g., 150 lbs), and height information (e.g., 5 feet, 9 inches) specific to the user, as well as user preferences (e.g., privacy settings).

Such information may be provided by the user and stored (e.g., on the memory of the user computing device) and/or server(s) 102. As shown, a user may provide his or her information via GUI 400, where the body imagery app may ask for, by way of non-limiting example, any one or more name, email, sex, height, weight, etc. It is to be understood that additional or different body data may also be captured or entered. The body data may be combined with the digital images (e.g., digital image 202a) captured for the user as described herein, which allows determination of user-specific measurements and/or health type-based identification of the user.

In some aspects, body data may comprise one or more of the following: sex, age, weight, weight differential over a period of time, height, height differential over a period of time, body mass index (BMI), BMI differential over a period of time, body fat percentage, body fat percentage differential over a period of time, body muscle percentage, body muscle percentage differential over a period of time, fitness information, health information, apparel information. Fitness information may comprise information regarding the user's fitness, such as how often they exercise, how long they exercise, what kind of exercise they do (e.g., cardio, calisthenics, weight lifting, gymnastics, golf, etc.), personal bests (e.g., max weight lifted, lowest time running a mile, etc.), etc. Health information may comprise any information regarding the user's health, such as medical history, family history, prescribed pharmaceuticals, etc. Apparel information may comprise information regarding the apparel/clothing present (e.g., shirt, yoga pants, hat, shoes, jewelry) in one or more digital images of the user (e.g., digital image 202a) and/or clothing not present in the one or more digital images of the user (e.g., undergarment, brazier, brazier size, etc.) Body data may indicate how a characteristic (e.g., weight, height, etc.) of a user has changed over a period of time by, e.g., indicating the differential of that characteristic over a period of time. For example, body data may indicate a user's net weight has increased over a 3-month period of time. In another example, body data may indicate a user's weight for each week in a 3-month period. Furthermore, health information and/or fitness information may comprise any information regarding a user and their characteristics over a period of time.

In some aspects, user-specific measurements of one or more portions of the user's body may be determined based on the one or more digital images and apparel information indicating the apparel the user is wearing in the one or more digital images. Additionally, or alternatively, user-specific body-based confidence interval may be based on user-specific measurements and apparel information indicating the apparel present in the one or more digital images. For example, a user wearing e.g., tight clothing, loose fitting clothing, no clothing, accessories (e.g., hat, shoes, jewelry), may appear to have various body measurements depending on the apparel being worn. Accordingly, user-specific measurements and user-specific body-based confidence intervals may be based on the apparel information.

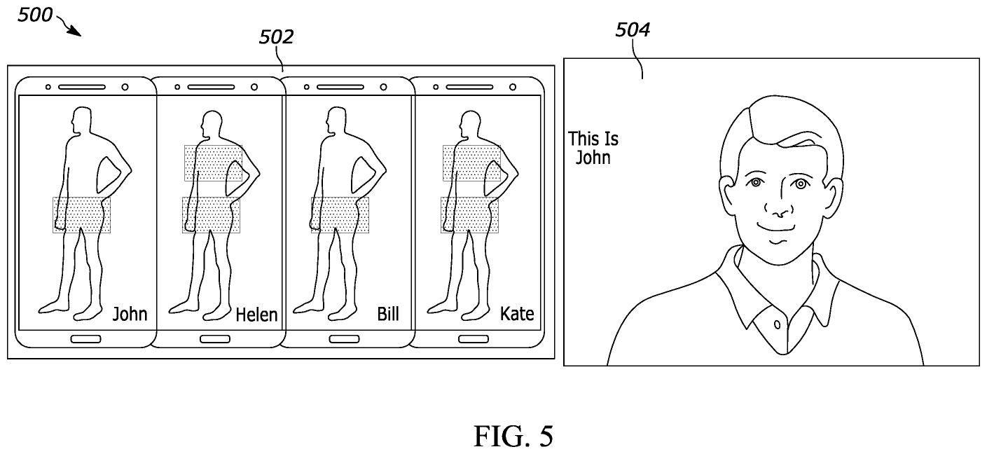

FIG. 5 illustrates an example graphic user interface (GUI) as rendered on a display screen of a user computing device regarding virtual avatar(s) 502 in accordance with various aspects disclosed herein. GUI 500 may be implemented or rendered in the same manner as described herein for GUI 300 of FIG. 3, where, for example, the body imagery app renders GUI 500 on a display screen via of a user computing device (e.g., user computing device 111c1).

As shown for FIG. 5, GUI 500 may render a virtual avatar such as any of the virtual avatars 502 as shown, including a first virtual avatar (“John”), a second virtual avatar (“Helen”), a third virtual avatar (“Bill”), and a fourth virtual avatar (“Kate”). A virtual avatar may be referred to a “TAV” herein, and it is to be understood that additional and/or different avatars may be generated, one for a given, specific user at one or more moments in time. That is, each of the virtual avatars 502 may represent different users and their respective measurements as determined by the digital imaging systems and methods as described herein. That is, from the user's respective sizing profile and body measurements (e.g., user-specific measurements), a unique avatar may be generated representing their physique, allowing the user to virtually observe, inspect, and/or assess portions of their body and/or their body as a whole. In some aspects, a user's unique avatar can be unisex. In some aspects, as shown for virtual avatars 504, the private or personal areas of the user maybe blurred, cropped, or obscured, e.g., for security reasons.

In other aspects, the user's virtual avatar can be rendered as a realistic avatar 504 (e.g., “John”), detailing the actual user in virtual space in order to provide an enhanced degree of detail and replication for virtually observing physical characteristics of the user. Such realistic rendering provides enhanced detail with respect to a realistic approach of observing a user's physical characteristics by observing the avatar representing the scanned user.

As illustrated for FIG. 5, in various aspects, a virtual avatar (e.g., any one of virtual avatars 502) may be generated for a user based on the user's user-specific measurements. The virtual avatar may be configured to depict one or more portions of the virtual avatar's body (e.g., upper body for virtual avatar 504) corresponding to the one or more portions of the user's body (e.g., in real life).

In some aspects, the virtual avatar may be rendered as a representation of the user. In some aspects, a virtual avatar may be generated for a user, as described herein, at a first time. A second virtual avatar may then be generated for the user at a second time. Each virtual avatar for the user may be based on the user-specific measurements at the first time and second time, respectively. The body imagery app or otherwise instructions operating at server(s) 102 may compare the second virtual avatar generated at the second time to the first virtual avatar captured at the first time to determine a user-specific body-based confidence interval for one or more of the one or more portions of the user's body. In this way, the digital systems and methods herein may be used for tracking, e.g., tracking fitness and/or health goals based on virtual avatar differences, tracking changes in user-specific body-based confidence intervals. That is, the comparison comprises scanning a user's body at the first time and the second time, where the comparison shows or demonstrates, for example, differences in body size and/or mass between the first and second time allowing for tracking changes of the user.

In some aspects, user-specific measurements may be determined a first time, and user-specific measurements for one or more portions of the user's body may be determined at a second time. The body imagery app or otherwise instructions operating at server(s) 102 may compare the user-specific measurements determined at the second time to the user-specific measurements determined at the first time to determine a user-specific body-based confidence interval for one or more of the one or more portions of the user's body.

FIG. 6 illustrates an example graphic user interface (GUI) as rendered on a display screen of a user computing device regarding a user-specific body-based confidence interval and health type-based identification, in accordance with various aspects disclosed herein. GUI 600 and GUI 650 may be implemented or rendered in the same manner as described herein for GUI 300 of FIG. 3, where, for example, the body imagery app renders GUI 600 on a display screen via of a user computing device (e.g., user computing device 111c1).

As shown for FIG. 6 in GUI 600, a user-specific body-based confidence interval 604 is shown for a specific body portion 606 of the virtual avatar 602 which comprises one or more body portions. In the example of FIG. 6, the specific body portion 606 is shown as a torso. As shown in GUI 650, a health type-based identification 654 of the user is shown based on user-specific body-based confidence intervals 652 for one or more body portions. In the example of FIG. 6, the health type-based identification 654 is shown as obese, and the one or more body portions are shown as arm(s), torso, leg(s), chest, and glutes.

In some aspects, a virtual avatar (e.g., virtual avatar 602) may be configured to depict a user-specific body-based confidence interval (e.g., user-specific body-based confidence interval 604) for one or more of the one or more portions (e.g., specific body portion 606) of the virtual avatar's body, the user-specific body-based confidence interval for the one or more of the one or more portions of the virtual avatar's body corresponding to the user-specific body-based confidence interval for the one or more portions of the user's body. For example, a user-specific body-based confidence interval (e.g., 90.74%) may be rendered with, within, near, and/or next to the specific body portion 606 of the virtual avatar 602.

In some aspects, a health type-based identification of a user may be generated based on their user-specific body-based confidence interval (i.e., their “B Fit” as referred to herein) for one or more body portions. The health type-based identification may be selected from one or more predefined health types, as described elsewhere herein.

In various aspects, a user-specific body-based confidence interval (“B Fit”) is a confidence interval from 0-100%, which informs a user of how accurate or confident the prediction for (i) a body portion (e.g., a torso, such as body portion 606), (ii) one or more body portions, and/or (iii) a proportion of one or more body portions, indicate(s) a health issue. A score of 100% is of utmost confidence for a body portion is indicative of a health issue. A score of 0% represents no confidence. The percentage scores of a user-specific body-based confidence interval (“B Fit”) are generated by comparing the user's user-specific measurements (from the user's sizing profile) and body data. The user-specific body-based confidence interval (“B Fit”) may also be based on comparing user-specific measurements to user-specific measurements, such as when comparing a first body portion to a second body portion, as discussed elsewhere herein. Based on this information, the user-specific body-based confidence interval (“B Fit”) is generated to indicate how accurate or confident the prediction for a body portion indicates a health issue. In this way, the user-specific body-based confidence interval (“B Fit”) establishes a universal sizing approach where the health of a user is based on the user's dimensions and not merely on one-dimensional metrics (such as weight, height, body mass index (BMI)) which provide limited indication of a user's mass distribution (e.g., a user's dimensions, a dimension of a user's specific body portion, a dimension of a user's specific first body portion compared to a dimension of a user's second body portion, etc.). For each body portion, a user-specific body-based confidence interval (“B Fit”) may indicate a confidence in a body portion being indicative of a health issue. This enables informed decision making while observing physical attributes of a user. Similarly, for each body portion a user-specific body-based confidence interval (“B Fit”) may indicate a confidence in a body portion not being indicative of a health issue. For example, a B Fit of 5.6%, may indicate nearly no confidence in the body portion being indicative of a health issue, such as obesity, and therefore, the B Fit may indicate a confidence in the body portion not being indicative of the health issue.

As shown in the example of FIG. 6, user-specific body-based confidence interval 604 (“Your B Fit”) is rendered on GUI 600 for body portion 606. As shown, user-specific body-based confidence interval 604 indicates that there is a 90.74% chance that the body portion 606 is indicative of a health issue. This prediction is made on the user's user-specific measurements and body data as provided by the user and/or as captured by the digital imaging systems and methods as described herein. As further shown in the example of FIG. 6, health type-based identification 654 (“Your Health Type Identification”) is rendered on GUI 650 for the user. As shown, health type-based identification 654 indicates the user is identified as “Obese.” This prediction is made based on the user-specific body-based confidence interval for one or more of the one or more portions of the user's body. In the example of FIG. 6, the prediction is based on the user-specific body-based confidence interval 652 (“74.40”, “90.74”, “79.12”, “81.21”, “63.06”) for body portions (“Arm(s)”, “Torso”, Leg(s)”, “Chest”, Glutes”), respectively. In other words, the user-specific body-based confidence interval for the body portion arm(s), for example, indicates there is a 74.40% chance that the body portion (arm(s)) is indicative of a health issue, and this user-specific body-based confidence interval, in addition to the remaining user-specific body-based confidence intervals of the user-specific body-based confidence interval 652, are used to generate the health type-based identification 654 of the user, wherein the health type-based identification 654 is selected from one or more predefined health types.

In some aspects, the user-specific body-based confidence interval indicates a confidence interval for the prediction that one or more of the one or more portions of the user's body indicates a health issue. In various aspects, the health issue may comprise one or more of the following: underweight, healthy weight, overweight, obese, severely obese, pre-diabetic, diabetic, biological age is less than current age, biological age is greater than current age, high mortality rate, low mortality rate. For example, the health issue of “biological age is greater than current age” may be a health issue for an individual who's body (i.e., physiology) is functioning poorly when compared to how the individual's body would be expected to function at the calendar age of the individual. In another example, the health issue of “high mortality rate” may be a health issue for an individual who belongs to a population of similar individuals (based on, e.g., user-specific measurements, body data, etc.), wherein within that population the mortality rate (and/or rate of co-morbidities) is above a threshold. The threshold may be, e.g., a mortality rate equal to or greater than 10 deaths per 1,000 individuals per year may be considered a high mortality rate. In some aspects, a health issue may be a state of health of the user.

In some aspects, the user-specific body-based confidence interval is determined via a health identification artificial intelligence (AI) model. The health identification AI model may be trained with pixel data of a plurality of training images of individuals and respective body data of the respective individuals, and configured to output (i) the user-specific body-based confidence interval for one or more of the one or more portions of the user's body, and/or (ii) the health type-based identification of the user. Additionally, or alternatively, the health identification AI model may be trained with a plurality of user-specific measurements.

In some aspects, the health type-based identification of the user is generated via a health identification AI model. The health identification AI model may be trained with a plurality of user-specific body-based confidence intervals for a plurality of individuals and configured to output the health type-based identification of the user.

In some aspects, the user-specific measurements may be determined via an AI model trained with pixel data of a plurality of training images of a plurality of individuals and a plurality of respective user-specific measurements of the one or more portions of the respective individuals' body(ies).

In the example of FIG. 6, the health type-based identification 654 of the user is shown to be “obese”; however, as described herein, “obese” may be one of many predefined health types to be selected as a health type-based identification. In various aspects, the predefined health types may comprise (i) specific medical conditions of disease, illness, or injury (e.g., obesity, overweight, underweight, kyphosis, Marfan syndrome, low mobility, alcoholism, etc.), (ii) specific medical conditions the user is at risk for (e.g., co-morbidities of specific medical conditions, such as those of group (i), diabetes, heart disease, stroke, etc.), (iii) specific medical procedures the user is at risk for (e.g., knee replacement, corrective surgery, liver transplant, rehabilitation), (iv) specific medical conditions without disease, illness, or injury (e.g., healthy), (v) a likelihood of reaching an age and/or age range (e.g., 50% chance of reaching age 80, 95% chance of living 10-15 more years, etc.), (vi) a metric (e.g., percentage, years) of time decreased from an expected lifespan (e.g., 2 years off of life, 5-10 years off of life, 2% off of life), and/or (vii) undetermined (such as when the user-specific confidence intervals do not indicate any other predefined health types).

Each of the one or more predefined health types may comprise one or more sub-health types indicating e.g., the risk (e.g., likelihood, chance) of the user having and/or acquiring the health type. This may include indications the user may presently have the health type, acquire the health type in the future, and/or acquire the health type by a certain age (and/or age range). Sub-health types may also, for example, be other predefined health types, as described herein, associated with the predefined health type. Accordingly, the user-specific body-based confidence interval may indicate a confidence of a body portion being indicative of the health type-based identification e.g., obesity, and one or more sub-health type-based identifications e.g., knee replacement surgery, diabetes, likelihood of living 5 more years, etc. In these ways, in some aspects, one or more health type-based identifications may be generated.

For example, a user may have a large abdomen and otherwise slim figure. The user's user-specific measurements may indicate the user's stomach (belly, gut, torso) (i.e., body portion) is a certain proportion to another body portion(s) and/or body data (e.g., height, weight) and the determined user-specific body-based confidence interval for the stomach may be 95%. In this example, the user-specific body-based confidence interval may be based on the user-specific measurements and the body data. Specifically, the user-specific body-based confidence interval for the stomach body portion may be based on, for example, (a) the proportion of the stomach body portion to another body portion (e.g., chest, leg) being within, e.g., the 98th percentile of users, (b) the proportion of the stomach body portion to body data (e.g., height, weight) being above (or below) a threshold, (c) the size of the stomach body portion being, e.g., above a threshold and a value(s) of another body portion (e.g., head) and/or body data (e.g., fitness information (such as the number of days of 30 minutes of activity or more a week), health information (such as high blood pressure), etc.) being above and/or below a threshold, (d) the shape of the stomach being e.g., bulbous (as indicated by user-specific measurements and/or the virtual avatar) instead of being, e.g., irregularly shaped (such as caused by layers of skin and fat overlapping the other), and/or (d) output of a machine learning (ML) and/or artificial intelligence (AI) model trained on user-specific measurements, body data, user-specific body-based measurements, and/or health type-based identification. Such bases described above are exemplary and may be applied analogously to any body portion(s) of a user.

Continuing the above example, the health type-based identification of the user may be “fatty liver disease”. The “fatty liver disease” health type-based identification may comprise a sub-health type-based identification of “50% chance within the next 5 years”. In some aspects, one or more health type-based identifications may be generated. Continuing the above example, the health type-based identification of the user may be “enlarged abdomen” and “fatty liver disease”. In other words, the user's stomach body portion is 95% indicative of an “enlarged abdomen” and “fatty liver disease”. The “enlarged abdomen” health type-based identification may include sub-health types, such as hepatic cirrhosis, obesity, knee replacement surgery, and/or decreased life span of 5 years.

In some aspects, the health type-based identification of the user indicates one or more of the following: (i) a Body Mass Index (BMI) value, (ii) a diabetes diagnosis value, (iii) a mortality rate, (iv) a biological age value. In various aspects a BMI value may be a BMI value based on the standard methods of determining a user's BMI according to their weight (w) and height (h). The standard method of determining a user's BMI may be represented formulaically as e.g., (BMI=w/h²). Alternatively, or additionally, a BMI value may be a smart BMI value based on user-specific measurements and/or body data. In various aspects wherein the health type-based identification of the user indicates a BMI value based on user-specific measurements and/or body data (i.e., a smart BMI value), the inaccuracies of BMI values based on standard methodology may be avoided because e.g., complex characteristics of a user, such as body portion shape, body weight distribution, body proportion(s), body fat/muscle percentage, etc., are not reduced down to two metrics (weight and height) to inform a user's BMI value.

In some aspects, a smart BMI value may be determined by an AI model trained on a plurality of digital images of a plurality of individuals, a plurality of respective user-specific measurements, and/or a plurality of respective smart BMI values, the AI model configured to output smart BMI value(s) based on one or more digital images of a user and/or a plurality of user-specific measurements.

In various aspects a diabetes diagnosis value may indicate a likelihood of the user being severely diabetic, diabetic, pre-diabetic, and/or at risk of acquiring a diabetes of any kind in the future. For example, a diabetes diagnosis value between zero and nine may indicate the user is likely not diabetic, between 10 and 19 may indicate the user is likely pre-diabetic, and between 20 and 30 may indicate the user is likely diabetic. The diabetes diagnosis value may be based on user-specific measurements and/or body data. For example, user-specific measurements over a three-year period may indicate an acute increase in the circumference of one or more portions of a user's body. In this example, the user-specific measurements may be a clear indication of risk for diabetes, and a diabetes diagnosis value may be based on the user-specific measurements. In some aspects, a diabetes diagnosis value may be determined by an AI model trained on a plurality of digital images of a plurality of individuals, a plurality of respective user-specific measurements, and/or a plurality of respective diabetes diagnosis values, the AI model configured to output diabetes diagnosis value(s) based on one or more digital images of a user and/or a plurality of user-specific measurements.

In some aspects, a user-specific recommendation may be generated comprising a prediction to reduce or improve a health factor corresponding to the health type-based identification of the user. For example, in some aspects, a diabetes diagnosis value may comprise a value between 20 and 30 indicating that the user is likely diabetic. Accordingly, an output may be generated to recommend a diet and/or physical activity customized for the user and predicted to reduce or improve a health factor (e.g., the diabetes diagnosis value) corresponding to the health type-based identification of the user.

In various aspects, a mortality rate may be the death rate for other individuals in the same population as the user, the population based on e.g., user-specific measurements, body data, etc. In various aspects, a biological age value may indicate the calendar age at which the user's body is functioning based on e.g., user-specific measurements and/or body data, and regardless of the user's calendar age (amount of time from the user's date of birth to the current calendar date).

In some aspects, a user-specific body-based confidence interval for one or more of the one or more portions of the user's body may be based on user-specific measurements, body data, and a preselected health type-based identification. The preselected health type-based identification may be selected by the user and/or a third party (e.g., physician, insurance agent, a second user, etc.). By preselecting a health type-based identification, the user-specific body-based confidence interval may be determined for one or more of the one or more portions of the user's body to indicate a confidence of the body portion being indicative of the preselected health type health issue, regardless of if the preselected health type-based identification would or would not have been generated as the health type-based identification based on e.g., user-specific measurements and body data. This enables a user and/or third party to evaluate and/or understand the implications of the user's physical characteristics on their health, and thus more accurately observe a user's physical characteristics.

In some aspects, body imagery app (e.g., 108) may be configured to generate a health report of the user based on one or more of the following: the user-specific measurements of the one or more portions of the user's body, the user-specific body-based confidence interval for one or more of the one or more portions of the user's body, the health type-based identification of the user. The health report may be used to determine a medical or insurance policy of the user. For example, the health report may be based on a health type-based identification of “obese” and an insurance entity may determine a life insurance policy of the user based on the health report.

In some aspects, GUI 600 may be configured to receive a selection to purchase the medical or insurance policy determined based on the generated health report of the user. Continuing the above example, the insurance entity may transmit the details (e.g., price, conditions, etc.) of the life insurance policy to the user (e.g., via the body imagery app 108, via user device 111c1, etc.) and the user may want to purchase the life insurance policy. In this example, GUI 600 may be configured to receive a selection to purchase the life insurance policy.

### Additional Considerations

Although the disclosure herein sets forth a detailed description of numerous different aspects, it should be understood that the legal scope of the description is defined by the words of the claims set forth at the end of this patent and equivalents. The detailed description is to be construed as exemplary only and does not describe every possible aspect since describing every possible aspect would be impractical. Numerous alternative aspects may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain aspects are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example aspects, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example aspects, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example aspects, the processor or processors may be located in a single location, while in other aspects the processors may be distributed across a number of locations.

The performance of certain of the operations may be distributed among the one or more processors, not only residing within a single machine, but deployed across a number of machines. In some example aspects, the one or more processors or processor-implemented modules may be located in a single geographic location (e.g., within a home environment, an office environment, or a server farm). In other aspects, the one or more processors or processor-implemented modules may be distributed across a number of geographic locations.

This detailed description is to be construed as exemplary only and does not describe every possible aspect, as describing every possible aspect would be impractical, if not impossible. A person of ordinary skill in the art may implement numerous alternate aspects, using either current technology or technology developed after the filing date of this application.

Those of ordinary skill in the art will recognize that a wide variety of modifications, alterations, and combinations can be made with respect to the above described aspects without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed is:

1. A digital imaging method for detecting user-specific body imagery, the digital imaging method comprising:
obtaining, by one or more processors, one or more digital images of a user, each of the one or more digital images depicting one or more portions of the user's body;
obtaining, by the one or more processors, body data specific to the user;
determining, by a body imagery application (app) executing on the one or more processors, user-specific measurements of the one or more portions of the user's body based on the one or more digital images;
determining, by the body imagery app, a user-specific body-based confidence interval for one or more of the one or more portions of the user's body, the user-specific body-based confidence interval based on the user-specific measurements and the body data; and
generating a health type-based identification of the user, based on the user-specific body-based confidence interval for one or more of the one or more portions of the user's body, wherein the health type-based identification is selected from one or more predefined health types.

2. The digital imaging method of claim 1, wherein the one or more portions of the user's body comprises a first body portion and a second body portion, and the user-specific body-based confidence interval is based on a proportion identified within pixel data of the one or more digital images between the first body portion and the second body portion.

3. The digital imaging method of claim 2, wherein the first body portion is a torso and the second body portion is a leg.

4. The digital imaging method of claim 2, wherein the user-specific body-based confidence interval is based on (i) a proportion identified within the pixel data between the first body portion and the second body portion, and (ii) the body data.

5. The digital imaging method of claim 1, wherein the one or more portions of the user's body comprises a first body portion, and the user-specific body-based confidence interval is based on a proportion identified within pixel data between the first body portion and one or more of the one or more portions of the user's body.

6. The digital imaging method of claim 1, wherein body data comprises one or more of the following: sex, age, weight, weight differential over a period of time, height, height differential over a period of time, body mass index (BMI), BMI differential over a period of time, body fat percentage, body fat percentage differential over a period of time, muscle percentage, muscle percentage differential over a period of time, fitness information, health information, apparel information.

7. The digital imaging method of claim 1, wherein the user-specific measurements of the one or more portions of the user's body comprise one or more of the following: width, height, length, circumference, volume.

8. The digital imaging method of claim 1 further comprising:
generating a user profile of the user based on the one or more digital images and the body data of the user.

9. The digital imaging method of claim 8 further comprising:
electronically transmitting the user profile to a second user.

10. The digital imaging method of claim 1 further comprising:
generating a virtual avatar for the user based on the user-specific measurements, the virtual avatar configured to depict one or more portions of the virtual avatar's body corresponding to the one or more portions of the user's body.

11. The digital imaging method of claim 10, wherein the virtual avatar is configured to depict a user-specific body-based confidence interval for one or more of the one or more portions of the virtual avatar's body, the user-specific body-based confidence interval for the one or more of the one or more portions of the virtual avatar's body corresponding to the user-specific body-based confidence interval for the one or more portions of the user's body.

12. The digital imaging method of claim 10, wherein the virtual avatar is rendered as a representation of the user.

13. The digital imaging method of claim 1 further comprising:
generating a health report of the user based on one or more of the following: the user-specific measurements of the one or more portions of the user's body, the user-specific body-based confidence interval for one or more of the one or more portions of the user's body, the health type-based identification of the user,
wherein the health report is used to determine a medical or insurance policy of the user.

14. The digital imaging method of claim 13 further comprising:
receiving a selection to purchase the medical or insurance policy determined based on the generated health report of the user.

15. The digital imaging method of claim 10:
wherein the virtual avatar is generated at a first time, and
wherein the digital imaging method further comprises:
generating a second virtual avatar for the user based on the user-specific measurements at a second time; and
comparing the second virtual avatar generated at the second time to the virtual avatar captured at the first time to determine a user-specific body-based confidence interval for one or more of the one or more portions of the user's body.

16. The digital imaging method of claim 1:
wherein the user-specific measurements are determined a first time, and
wherein the digital imaging method further comprises:
determining user-specific measurements for one or more portions of the user's body at a second time; and
comparing the user-specific measurements determined at the second time to the user-specific measurements determined at the first time to determine a user-specific body-based confidence interval for one or more of the one or more portions of the user's body.

17. The digital imaging method of claim 1:
wherein the user-specific body-based confidence interval is determined via a health identification artificial intelligence (AI) model, and
wherein the health identification AI model is trained with pixel data of a plurality of training images of individuals and respective body data of the respective individuals, the health identification AI model configured to output one or more of the following:
(i) the user-specific body-based confidence interval for one or more of the one or more portions of the user's body, and
(ii) the health type-based identification of the user.

18. The digital imaging method of claim 1:
wherein the health type-based identification of the user is generated via a health identification artificial intelligence (AI) model, and
wherein the health identification AI model is trained with a plurality of user-specific body-based confidence intervals for a plurality of individuals, the health identification AI model configured to output the health type-based identification of the user.

19. The digital imaging method of claim 1,
wherein the user-specific body-based confidence interval indicates a confidence interval for a prediction that one or more of the one or more portions of the user's body indicates a health issue, and
wherein the health issue comprises one or more of the following:
underweight, healthy weight, overweight, obese, severely obese, pre-diabetic, diabetic, biological age is less than current age, biological age is greater than current age, high mortality rate, low mortality rate.

20. The digital imaging method of claim 1:
wherein the health type-based identification of the user indicates one or more of the following:
a Body Mass Index (BMI) value,
a diabetes diagnosis value,
a mortality rate,
a biological age value.

21. The digital imaging method of claim 1 further comprising:
generating a user-specific recommendation comprising a prediction to reduce or improve a health factor corresponding to the health type-based identification of the user.

22. A digital imaging system configured to detect user-specific body imagery, the digital imaging system comprising:
a body imagery application (app) comprising computing instructions configured to execute on one or more processors,
wherein the computing instructions of the body imagery app when executed by the one or more processors, cause the one or more processors to:
obtain one or more digital images of a user, each of the one or more digital images depicting one or more portions of the user's body,
obtain body data specific to the user,
determine user-specific measurements of the one or more portions of the user's body based on the one or more digital images,
determine a user-specific body-based confidence interval for the user, the user-specific body-based confidence interval based on the user-specific measurements and the body data, and
generate a health type-based identification of the user that corresponds to one or more predefined health types based on one or more of the user-specific body-based confidence interval.

23. The digital imaging system of claim 22, wherein the one or more portions of the user's body comprises a first body portion and a second body portion, and the user-specific body-based confidence interval is based on a proportion identified within pixel data of the one or more digital images between the first body portion and the second body portion.

24. The digital imaging system of claim 23, wherein the first body portion is a torso and the second body portion is a leg.

25. The digital imaging system of claim 23, wherein the user-specific body-based confidence interval is based on (i) a proportion identified within pixel data of the one or more digital images between the first body portion and the second body portion, and (ii) the body data.

26. The digital imaging system of claim 22, wherein the one or more portions of the user's body comprises a first body portion, and the user-specific body-based confidence interval is based on a proportion identified within pixel data of the one or more digital images between the first body portion and one or more of the one or more portions of the user's body.

27. The digital imaging system of claim 22, wherein body data comprises one or more of the following: sex, age, weight, weight differential over a period of time, height, height differential over a period of time, body mass index (BMI), BMI differential over a period of time, body fat percentage, body fat percentage differential over a period of time, body muscle percentage, body muscle percentage differential over a period of time, fitness information, health information, apparel information.

28. The digital imaging system of claim 22, wherein the user-specific measurements of the one or more portions of the user's body comprise one or more of the following: width, height, length, circumference, volume.

29. The digital imaging system of claim 22, wherein the computing instructions of the body imagery app when executed by the one or more processors, further cause the one or more processors to:
generate a user profile of the user based on the one or more digital images and the body data of the user.

30. The digital imaging system of claim 27, wherein the computing instructions of the body imagery app when executed by the one or more processors, further cause the one or more processors to:
electronically transmit the user profile to a second user.

31. The digital imaging system of claim 22, wherein the computing instructions of the body imagery app when executed by the one or more processors, further cause the one or more processors to:
generate a virtual avatar for the user based on the user-specific measurements, the virtual avatar configured to depict one or more portions of the virtual avatar's body corresponding to the one or more portions of the user's body.

32. The digital imaging system of claim 31, wherein the virtual avatar is configured to depict a user-specific body-based confidence interval for one or more of the one or more portions of the virtual avatar's body, the user-specific body-based confidence interval for the one or more of the one or more portions of the virtual avatar's body corresponding to the user-specific body-based confidence interval for the one or more portions of the user's body.

33. The digital imaging system of claim 31, wherein the virtual avatar is rendered as a representation of the user.

34. The digital imaging system of claim 22, wherein the computing instructions of the body imagery app when executed by the one or more processors, further cause the one or more processors to:
generate a health report of the user based on one or more of the following: the user-specific measurements of the one or more portions of the user's body, the user-specific body-based confidence interval for one or more of the one or more portions of the user's body, the health type-based identification of the user,
wherein the health report is used to determine a medical or insurance policy of the user.

35. The digital imaging system of claim 34, wherein the computing instructions of the body imagery app when executed by the one or more processors, further cause the one or more processors to:
receive a selection to purchase the medical or insurance policy determined based on the generated health report of the user.

36. The digital imaging system of claim 31, wherein the virtual avatar is generated a first time, and wherein the computing instructions of the body imagery app when executed by the one or more processors, further cause the one or more processors to:
generate a second virtual avatar for the user based on the user-specific measurements at a second time; and
compare the second virtual avatar generated at the second time to the virtual avatar captured at the first time to determine a user-specific body-based confidence interval for one or more of the one or more portions of the user's body.

37. The digital imaging system of claim 22, wherein the user-specific measurements are determined a first time, and wherein the computing instructions of the body imagery app when executed by the one or more processors, further cause the one or more processors to:
determine user-specific measurements for one or more portions of the user's body at a second time; and
compare the user-specific measurements determined at the second time to the user-specific measurements determined at the first time to determine a user-specific body-based confidence interval for one or more of the one or more portions of the user's body.

38. The digital imaging system of claim 22, further comprising a health identification artificial intelligence (AI) model trained with pixel data of a plurality of training images of individuals and respective body data of the respective individuals, the health identification model configured to output one or more of the following:
(i) a user-specific body-based confidence interval for one or more of the one or more portions of the user's body, and
(ii) the health type-based identification of the user; and
wherein the computing instructions of the body imagery app when executed by the one or more processors, further cause the one or more processors to do one or more of the following:
(i) determine, by the health identification AI model based on the one or more digital images of the user and the body data specific to the user, the user-specific body-based confidence interval for one or more of the one or more portions of the user's body, and
(ii) generate, by the health identification AI model based on the one or more digital images of the user and the body data specific to the user, the health type-based identification of the user.

39. The digital imaging system of claim 22, further comprising a health identification artificial intelligence (AI) model trained with a plurality of user-specific body-based confidence intervals for a plurality of individuals, the health identification model configured to output the health type-based identification of the user, and wherein the computing instructions of the body imagery app when executed by the one or more processors, further cause the one or more processors to:
generate, by the health identification AI model based on the user-specific body-based confidence interval of the user, the health type-based identification of the user.

40. The digital imaging system of claim 22, wherein the user-specific body-based confidence interval indicates a confidence interval for a prediction that one or more of the one or more portions of the user's body indicates a health issue, and
wherein the health issue comprises one or more of the following:
underweight, healthy weight, overweight, obese, severely obese, pre-diabetic, diabetic, biological age is less than current age, biological age is greater than current age, high mortality rate, low mortality rate.

41. The digital imaging system of claim 22, wherein the health type-based identification of the user indicates one or more of the following:
a Body Mass Index (BMI) value,
a diabetes diagnosis value,
a mortality rate,
a biological age value.

42. The digital imaging system of claim 41, wherein the computing instructions of the body imagery app when executed by the one or more processors, further cause the one or more processors to:
generate a user-specific recommendation predicted to reduce or improve a health factor corresponding to the health type-based identification of the user.

43. The digital imaging system of claim 41, wherein the computing instructions of the body imagery app when executed by the one or more processors, further cause the one or more processors to:
generate a user-specific recommendation comprising a prediction to reduce or improve a health factor corresponding to the health type-based identification of the user.

44. A tangible, non-transitory computer-readable medium storing instructions for detecting user-specific body imagery, that when executed by one or more processors cause the one or more processors to:
obtain one or more digital images of a user, each of the one or more digital images depicting one or more portions of the user's body;
obtain body data specific to the user;
determine user-specific measurements of the one or more portions of the user's body based on the one or more digital images;
determine a user-specific body-based confidence interval for one or more of the one or more portions of the user's body, the user-specific body-based confidence interval based on the user-specific measurements and the body data; and
generate a health type-based identification of the user that corresponds to one or more predefined health types based on one or more of the user-specific body-based confidence interval.
